(19) United States
(12) Patent Application Publication
(10) Pub. No.: US 2012/0195979 A1
(43) Pub. Date: Aug. 2, 2012

(54) TREATMENT COMPOSITION FOR RECREATIONAL WATER

(75) Inventors: Derek Francis PARISH, Peachtree City, GA (US); Christopher M. ZETENA, Atlanta, GA (US)

(73) Assignee: ARCH CHEMICALS, INC., Norwalk, CT (US)

(21) Appl. No.: 13/446,366

(22) Filed: Apr. 13, 2012

Related U.S. Application Data
(62) Division of application No. 11/807,051, filed on May 25, 2007.
(60) Provisional application No. 60/810,098, filed on Jun. 1, 2006.

(51) Int. Cl. A61K 31/08; A61K 33/22; A61K 33/04; A61K 33/06; A61K 38/16; A61P 17/16; C02F 1/68; A61K 31/195; C02F 103/42

(52) U.S. Cl. 424/660; 210/749; 210/754; 210/764; 514/723; 424/715; 424/717; 424/709; 514/566; 424/682; 514/18.8

(57) ABSTRACT

The present invention is directed to a treatment composition for recreational water, comprising a high molecular weight water soluble resin composition made from long chain alkoxylated polymers or co-polymers that impart emollient properties to the recreational water, as well as articles of manufacture comprising packaging material and the treatment composition for recreational water contained within the packaging material. The present invention is also directed to methods for treating recreational water, and recreational water treated with the above treatment composition.

### CROSS-REFERENCE TO RELATED APPLICATIONS

This application is a division of U.S. patent application Ser. No. 11/807,051 filed May 25, 2007, which in turn claims the benefit under 35 U.S.C. §119(e) of U.S. Provisional patent application Ser. No. 60/810,098 filed Jun. 1, 2006. The disclosure of both applications are incorporated herein by reference.

### BACKGROUND OF THE INVENTION

1. Field of the Invention

The present invention is directed to treatment compositions for treating recreational water, and in particular to treatment compositions for recreational water that impart emollient properties to the recreational water.

2. Brief Description of the Related Art

A variety of chemicals are used to treat recreational water, such as swimming pools, spas, hot tubs, and the like. These chemicals can typically be divided into two groups. The first group may be broadly classified as sanitizers which function to keep the water free from microorganisms. Examples of such chemicals include calcium hypochlorite, trichlor, bromine, and quaternary ammonium compounds, and are available commercially. These chemicals serve to eradicate or control deleterious amounts of pathogenic bacteria, algae, and other microorganisms that are present in aqueous media. The second group of chemicals are used to adjust the water chemistry to make it suitable for recreational use, and to make it less likely to cause damage to the hardware of the swimming pool. Examples of chemicals in this second group include acids, bases, buffers, and other chemicals which function to adjust and maintain the pH and other characteristics of the chemistry of the pool water.

While the above chemicals do an excellent job of sanitizing the water and adjusting its chemistry, one disadvantage of these chemicals is their harsh treatment of the bather's skin and hair. For example, one undesired side-effect of the use of these chemicals is gradual depletion of oils and natural moisturizing factors from the swimmer or bather's skin. This effect goes unnoticed while in the pool or spa, but manifests itself by causing the swimmer or bather's skin to feel dry after a short period of time after exiting the pool or spa. In addition, these chemicals can dry out and damage the bather's scalp and hair, leaving the hair brittle and dull.

U.S. Pat. Nos. 4,162,990 and 4,250,140 to Rowlette disclose methods and compositions for retarding evaporation of water from open water sources such as pools and spas. The compositions disclosed in these patents are a combination of a saturated aliphatic hydrocarbon such as docosanol and docosanoic acid, and a spreading agent such as polyethylene glycol.

What is needed in the art is a product for treatment of recreational water that prevents, diminishes, or counteracts the adverse effects of regular water treatment chemicals to the bather's skin and hair. The present invention is believed to be an answer to that need.

### SUMMARY OF THE INVENTION

In one aspect, the present invention is directed to a treatment composition for recreational water, comprising a high molecular weight water soluble resin composition comprising long chain alkoxylated polymers or co-polymers that imparts emollient properties to said recreational water.

In another aspect, the present invention is directed to a treatment composition for recreational water, comprising a high molecular weight water soluble resin composition comprising long chain alkoxylated polymers or copolymers selected from the group consisting of polyethylene oxide, polyethylene glycol, polypropylene glycol, polybutylene glycol, and combinations thereof, and additives selected from the group consisting of sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof; wherein said long chain alkoxylated polymers or copolymers have molecular weights between about 1 million to about 8 million, and wherein said treatment composition imparts emollient properties to said recreational water.

In another aspect, the present invention is directed to an article of manufacture comprising packaging material and a treatment composition for recreational water contained within said packaging material, wherein said packaging material comprises a label which indicates that said treatment composition imparts emollient properties to said recreational water, and wherein said treatment composition comprises a high molecular weight water soluble resin composition comprising long chain alkoxylated polymers or copolymers selected from the group consisting of polyethylene oxide, polyethylene glycol, polypropylene glycol, polybutylene glycol, and combinations thereof, and wherein said high molecular weight polymers have molecular weights between about 1 million to about 8 million.

In another aspect, the present invention is directed to an article of manufacture comprising packaging material and a treatment composition for recreational water contained within said packaging material, wherein said packaging material comprises a label which indicates that said treatment composition imparts emollient properties to said recreational water, and wherein said treatment composition comprises a high molecular weight water soluble resin composition comprising long chain alkoxylated polymers or copolymers selected from the group consisting of polyethylene oxide, polyethylene glycol, polypropylene oxide, polybutylene oxide, and combinations thereof, and additives selected from the group consisting of sodium tetraborate, sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof, and wherein said long chain alkoxylated polymers or copolymers have molecular weights between about 1 million to about 8 million.

In another aspect, the present invention is directed to a method for treating recreational water, comprising the step of: combining recreational water with a treatment composition that imparts emollient properties to said recreational water, said treatment composition comprising a high molecular weight water soluble resin composition comprising long chain alkoxylated polymers or copolymers in an amount sufficient to provide a concentration of said long chain alkoxylated polymers or copolymers in said recreational water of between 10 and 1000 ppm.

In another aspect, the present invention is directed to a method for treating recreational water to have emollient properties, comprising the step of: combining recreational water with a treatment composition that imparts emollient properties to said recreational water, said treatment composition comprising a high molecular weight water soluble resin composition comprising long chain alkoxylated polymers or copolymers selected from the group consisting of polyethylene oxide, polyethylene glycol, polypropylene glycol, polybutylene glycol, and combinations thereof, and additives selected from the group consisting of sodium tetraborate, sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof, in an amount sufficient to provide a concentration of said long chain alkoxylated polymers or copolymers in said recreational water of between 10 and 1000 ppm, and wherein said treatment composition imparts emollient properties to said recreational water.

In another aspect, the present invention is directed to recreational water comprising from about 10 to about 1000 ppm of long chain alkoxylated polymers or copolymers that impart emollient properties to said recreational water.

These and other aspects will become apparent upon reading the following detailed description of the invention.

### DETAILED DESCRIPTION OF THE INVENTION

It has been unexpectedly discovered that high molecular weight water soluble resins can advantageously impart emollient properties to recreational water, and thus counteract the drying effect that results from bathing or swimming in recreational water that has been treated with common sanitizing or chemistry adjustment chemicals. The high molecular weight water soluble resins also impart a smooth feeling to the water when compared to untreated water, and makes for a more enjoyable bathing or swimming experience. The high molecular weight water soluble resins may be combined with other ingredients, such as conventional pool care chemicals, personal care ingredients, vitamins, and the like, to further enhance the water treatment.

As indicated above, the present invention is directed to methods and compositions for treating recreational water, comprising high molecular weight water soluble resins that impart emollient properties to the water. As defined herein, the phrase “recreational water” refers to bodies of water for leisure, sporting, or therapeutic purposes, such as swimming pools, spas, hot tubs, and the like. The term “emollient” refers to agents that soften, soothe, and/or moisturize the skin, and “emollient properties” refers to soothing, softening, and/or moisturizing characteristics that result from emollients being added to recreational water.

The treatment composition of the invention comprises a high molecular weight water soluble resin composition that imparts emollient properties to the recreational water. The high molecular weight water soluble resin composition is comprised of one or more types of high molecular weight water soluble polymers, and particularly long-chain alkoxylated polymers or copolymers. The term “high molecular weight” refers generally to polymers having molecular weights ranging preferably from about 1 million to about 8 million, more preferably from about 2 million to about 6 million, and most preferably from about 3 million to about 5 million. Examples of suitable high molecular weight water soluble polymers that may be used in the treatment composition of the invention include alkoxylated polymers or copolymers such as polyethylene oxide, polyethylene glycol, polypropylene glycol, polybutylene glycol, as well as combinations of two, three, four, or more of these polymers, including homopolymers and copolymers in all possible combinations. One particularly suitable high molecular weight water soluble polymer is polyethylene oxide having a molecular weight of about 4 million and sold under the tradename POLYOX WSR-301 by Dow Chemical Company (Midland, Mich.). Preferably, the high molecular weight water soluble resin comprises from about 50 to about 100% by weight of the treatment composition, and more preferably from 90 to about 99.9% by weight of the treatment composition.

The high molecular weight water soluble resin composition may be supplied as a fine free-flowing powder or as granular for inclusion in the water treatment composition of the invention. In general, solid, granular, tablet, or powder forms are preferred since their dissolution rates can be more easily controlled and the final product is easier to produce in unit dose form. As will be appreciated by those of skill in the art, the high molecular weight water soluble resin may also be added in the form of an aqueous solution. Regardless of form, a quantity of the high molecular weight water soluble resin is added to the treatment composition of the present invention so that the treated recreational water has a final concentration of high molecular weight water soluble polymers preferably in a range from between about 10 and 1000 ppm, more preferably from about 20 to about 100 ppm, and most preferably from between about 40 ppm and 50 ppm.

Additional optional ingredients may be added to the treatment composition of the invention to add additional characteristics to the water. Useful additional optional ingredients include chemicals used in the treatment of recreational water, or ingredients used in personal care compositions. Examples of suitable additional ingredients include sodium tetraborate, sodium carbonate, sodium bicarbonate, sodium bisulfate, ethylene diamine tetraacetic acid (EDTA), calcium chloride, powdered silicon antifoam, cationic polymers, quaternary ammonium compounds, mineral salts such as Dead Sea salts, or epsom salts, soy proteins, vitamins such as vitamin E, vitamin C and the like, fragrances, colorants, moisturizers such as Bioplex NMF sold by Arch Personal Care Products (South Plainfield, N.J.) and combinations thereof. Generally, these additional optional ingredients comprise from about 0 to about 90% by weight, and more preferably from about 0.1 to about 10% by weight, based on the total weight of the treatment composition. Preferably, these optional ingredients are added in amounts sufficient to provide final concentrations in the recreational water of from 0.5 to 500 ppm, and more preferably from 1 to 250 ppm. In one preferred embodiment, sodium tetraborate is added to the composition such that the concentration of sodium tetraborate in the water ranges from about 30 to about 50 ppm.

In one preferred embodiment, 60-80% by weight of sodium tetraborate is included as an additive that enhances the dissolution rate of the of high molecular weight water soluble polymers. One suitable source of sodium tetraborate for use in recreational water is sold by Arch Chemicals, Inc. (Norwalk, Conn.) under the trade name “ENDURE”. The time taken to dissolve polymer depends on at least two factors—amount of polymer added and water temperature. For example, when 600 g of granulated polymer is added to warm pool water (85° F.), it takes just under a day for the added polymer to completely dissove and for the flow rate to recover to the same rate prior to the addition of the polymer. However, it has been unexpectedly discovered that when 1500 g of a granulated 40% polymer/60% sodium tetraborate blend (equal to 600 g of polymer) is added to the same pool, the flow rate takes 5 hours to return to that prior to addition of the blend. The recovery in both instances was measured by following the flow rate and back pressure on the filter.

The treatment composition of the present invention is made using dry material mixing and blending techniques known in the art, and no special methods are required.

The treatment composition is preferably packaged as an article of manufacture for retail sale. In this embodiment, the treatment composition is contained within packaging material, and the packaging material includes a label or leaflet which indicates that the treatment composition imparts emollient properties to the recreational water. The packaging material may be a box, bag, or other material appropriate for retail sales use, and which can contain the treatment composition prior to use. The treatment composition contained within the packaging may be the high molecular weight water soluble resin composition described above, either alone or in combination with any or all of the above optional ingredients. The high molecular weight water soluble resin composition contained within the packaging may also be in any suitable form, for example, powders, granules, tablets, and the like.

In a related embodiment, the present invention is also directed to methods of treating recreational water using the above treatment compositions. In preferred embodiments, the treatment composition is added to the recreational water in an amount sufficient to provide a concentration of high molecular weight water soluble polymers in the recreational water of between 10 and 1000 ppm, more preferably from about 20 to about 100 ppm, and most preferably from between about 40 ppm and 50 ppm. The treatment composition may be added to the recreational water by any method, including broadcasting or direct addition, use of a suitable feeder or floater, or addition to the skimmer or other circulation equipment.

### EXAMPLES

The following examples are intended to illustrate, but in no way limit the scope of the present invention. All parts and percentages are by weight, and temperatures are in degrees Celsius unless explicitly stated otherwise.

The following Table outlines examples of dosage quantities of POLYOX WSR-301 (Dow Chemical Company, Midland Mich.) added to various amounts of pool water. Briefly, various weights of POLYOX WSR-301 (polyethylene oxide having a molecular weight of about 4 million) in powder form were added slowly to a pool skimmer in such a manner that a dry layer of powder does not build up. As the powder contacted the water, it was pulled into the skimmer and pool plumbing system, and dissolved rapidly. Following dissolution, samples of pool water were evaluated for final concentrations polymer, and for subjective assessments of emollient properties (e.g., soothing, softening, and/or moisturizing characteristics) relative to each other.

TABLE I

| | | | Final | |
|---|---|---|---|---|
| | Pool Volume | Amount | Concentration | Emollient |
| Example | (Gallons) | (lbs) | (ppm) | Properties |
| 1 | 10,000 | 0.83 | 10 | + |
| 2 | 10,000 | 1.66 | 20 | ++ |
| 3 | 10,000 | 2.50 | 30 | +++ |
| 4 | 10,000 | 3.50 | 40 | ++++ |
| 5 | 15,000 | 1.25 | 10 | + |
| 6 | 15,000 | 2.50 | 20 | ++ |
| 7 | 15,000 | 3.75 | 30 | +++ |
| 8 | 15,000 | 5.00 | 40 | ++++ |
| 9 | 20,000 | 1.66 | 10 | + |
| 10 | 20,000 | 3.32 | 20 | ++ |
| 11 | 20,000 | 5.00 | 30 | +++ |
| 12 | 20,000 | 7.00 | 40 | ++++ |

As shown in Table I, increasing amounts of polyethylene oxide to the pool water resulted in increasing desirable emollient properties (“+” being least desirable, and “++++” being most desirable), with the most desirable emollient properties occurring in pool water having final concentrations of about 40 ppm of polyethylene oxide.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

What is claimed is:

1. A method for treating recreational water, comprising combining recreational water with a treatment composition that imparts emollient properties to said recreational water, said treatment composition comprising a long chain alkoxylated polymer or copolymer in an amount sufficient to provide a concentration of said long chain alkoxylated polymer or copolymer in said recreational water of between 10 and 1000 ppm, wherein said treatment composition imparts emollient properties to said recreational water.

2. The method of claim 1, wherein said concentration of said long chain alkoxylated polymers or copolymers in said recreational water is between 20 and 100 ppm.

3. The method of claim 2, wherein said concentration of said long chain alkoxylated polymers or copolymers in said recreational water is between 40 and 50 ppm.

4. The method of claim 1, wherein said long chain alkoxylated polymers or copolymers are selected from the group consisting of polyethylene oxide, polyethylene glycol, polypropylene glycol, polybutylene glycol, and combinations thereof

5. The method of claim 1, wherein said treatment composition further comprises additives selected from the group consisting of sodium tetraborate, sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof

6. The method of claim 5, wherein said additives comprise from about 0 to about 90% by weight, based on the total weight of said treatment composition.

7. The method of claim 5, where said additives comprises 60-80% by weight of sodium tetraborate, based on the total weight of said treatment composition.

8. The method of claim 1, wherein said high molecular weight water soluble resin comprises from about 50 to about 100% by weight, based on the total weight of said treatment composition.

9. A method for treating recreational water to have emollient properties, comprising combining recreational water with a treatment composition that imparts emollient properties to said recreational water, said treatment composition comprising a high molecular weight water soluble resin composition comprising long chain alkoxylated polymers or copolymers selected from the group consisting of polyethylene oxide, polyethylene glycol, polypropylene glycol, polybutylene glycol, and combinations thereof, and additives selected from the group consisting of sodium tetraborate, sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof, in an amount sufficient to provide a concentration of said long chain alkoxylated polymers or copolymers in said recreational water of between 10 and 1000 ppm, and wherein said treatment composition imparts emollient properties to said recreational water.

10. The method of claim 9, wherein said concentration of said long chain alkoxylated polymers or copolymers in said recreational water is between 20 and 100 ppm.

11. The method of claim 10, wherein said concentration of said long chain alkoxylated polymers or copolymers in said recreational water is between 40 and 50 ppm.

12. The method of claim 9, wherein the concentration of said additives in said recreational water is between 0.5 and 500 ppm.

13. The method of claim 12, wherein the concentration of said additives in said recreational water is between 1 and 250 ppm.

14. The method of claim 9, wherein said additives comprise from about 0 to about 90% by weight, based on the total weight of said treatment composition.

15. The method of claim 9, where said additives comprises 60-80% by weight of sodium tetraborate, based on the total weight of said treatment composition.

16. The method of claim 9, wherein said high molecular weight water soluble resin comprises from about 50 to about 100% by weight, based on the total weight of said treatment composition.

17. Recreational water comprising from about 10 to about 1000 ppm of long chain alkoxylated polymers or copolymers that impart emollient properties to said recreational water.

18. The recreational water of claim 17, comprising from about 20 to about 100 ppm of said long chain alkoxylated polymers or copolymers.

19. The recreational water of claim 18, comprising from about 40 to about 50 ppm of said long chain alkoxylated polymers or copolymers.

20. The recreational water of claim 17, wherein said long chain alkoxylated polymers or copolymers are selected from the group consisting of polyethylene oxide, polyethylene glycol, polypropylene glycol, polybutylene glycol, and combinations thereof

21. The recreational water of claim 18, further comprising additives selected from the group consisting of sodium tetraborate, sodium carbonate, sodium bicarbonate, sodium bisulfate, ethylene diamine tetraacetic acid, calcium chloride, powdered silicon antifoam, cationic polymers, quaternary ammonium compounds, mineral salts, soy proteins, vitamins, fragrances, colorants, moisturizers, and combinations thereof, and wherein said additives comprise from 0.5 to 500 ppm.

22. The recreational water of claim 21, wherein said additives is 30-50 ppm of sodium tetraborate.
